(19) United States
(12) Patent Application Publication
(10) Pub. No.: US 2021/0353254 A1
(43) Pub. Date: Nov. 18, 2021

(54) METHODS AND SYSTEMS FOR AN INVASIVE DEPLOYABLE DEVICE

(71) Applicant: GE Precision Healthcare LLC, Milwaukee, WI (US)

(72) Inventors: Edouard Da Cruz, Nice (FR); Giandonato Stallone, Nice (FR); Flavien Daloz, Antibes (FR)

(21) Appl. No.: 15/930,302

(22) Filed: May 12, 2020

(51) Int. Cl. A61B 8/12; A61B 8/00

(52) U.S. Cl. CPC A61B 8/12; A61B 2562/225; A61B 2562/12; A61B 8/4494

(57) ABSTRACT

Various methods and systems are provided for a deployable invasive device. In one example, the deployable invasive device has a transducer including a plurality of transducer arrays spaced apart by a shape memory material. The shape memory material is configured to transition between a first configuration with a first set of dimensions and a second configuration with a second, larger set of dimensions.

### FIELD

Embodiments of the subject matter disclosed herein relate to a deployable invasive device.

### BACKGROUND

Invasive devices may be used to obtain information about tissues, organs, and other anatomical regions that may be difficult to gather via external scanning or imaging techniques. An invasive device may be a deployable catheter which may be inserted intravenously into a patient's body. In one example, the device may be used for intracardiac echocardiography imaging where the device is introduced into the heart via, for example, the aorta, inferior vena cava, or jugular vein. The devices may include an ultrasound probe with an aperture size conforming to dimensions that enable the devices to fit through an artery or vein. Thus a resolution and penetration of the ultrasound probe may be determined by a maximum allowable diameter of the invasive device.

### BRIEF DESCRIPTION

In one embodiment, a deployable invasive device comprises a transducer, including a plurality of transducer arrays spaced apart by a shape memory material, configured to transition between a first folded shape and a second unfolded shape. The transitioning of the transducer between the first and second shapes allows dimensions of the transducer to be modified in response to one or more stimuli. The transducer size may thereby be reduced to allow the transducer to pass through intravenous channels and increase when desired to obtain high resolution data with increased acquisition speed.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a block diagram of an exemplary imaging system including a deployable catheter.

FIG. 2 shows the deployable catheter of FIG. 1 in greater detail, including an exemplary imaging catheter tip and transducer for use in the system illustrated in FIG. 1.

FIG. 3 shows a first cross-sectional view of the exemplary imaging catheter tip which may be included in the deployable catheter of FIG. 2.

FIG. 4 is a schematic of a second cross-sectional view of the deployable catheter of FIG. 2.

FIG. 5 is a first diagram showing a two-way shape memory effect of a transducer incorporating a shape memory material.

FIG. 6A shows a first example of a transducer adapted with the shape memory material in a folded configuration.

FIG. 6B shows the first example of the transducer of FIG. 6A in an unfolded configuration.

FIG. 7A shows a second example of a transducer adapted with a shape memory material in a folded configuration.

FIG. 7B shows the second example of the transducer of FIG. 7A in an unfolded configuration.

FIG. 7C shows another view of the second example of the transducer of FIG. 7A in the folded configuration.

FIG. 8A shows a perspective view of the second example of the transducer of FIGS. 7A-7C in the folded configuration and enclosed in a balloon.

FIG. 8B shows an end view of the second example of the transducer in the folded configuration and enclosed in the balloon.

FIG. 9A shows a perspective view of the second example of the transducer of FIGS. 7A-7C in the unfolded configuration and enclosed in the balloon.

FIG. 9B shows an end view of the second example of the transducer in the unfolded configuration and enclosed in the balloon.

FIG. 10 shows a flow diagram of a process for fabricating and deploying a deployable catheter configured with a SMP.

FIG. 11 shows a second diagram depicting programming of a two-way shape memory SMP.

FIG. 12 shows a third diagram illustrating more than one type of shape transition of a SMP.

FIG. 13 shows an example of a method for fabricating a deployable catheter adapted with a SMP for intravenous imaging.

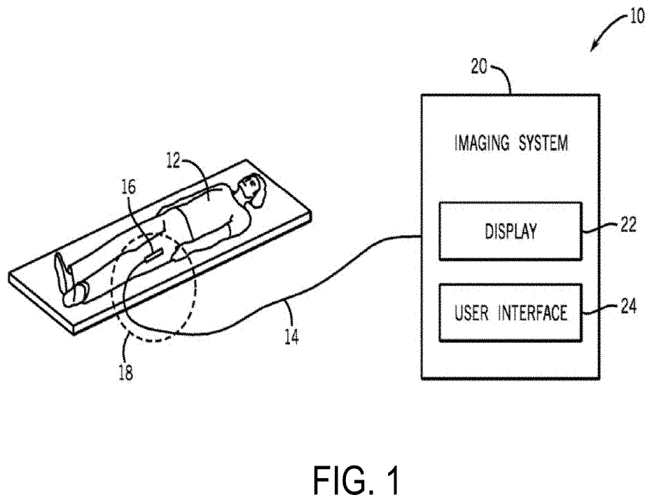

FIGS. 1-4 and 6A-9B are drawn approximately to scale although other relative dimensions may be used.

### DETAILED DESCRIPTION

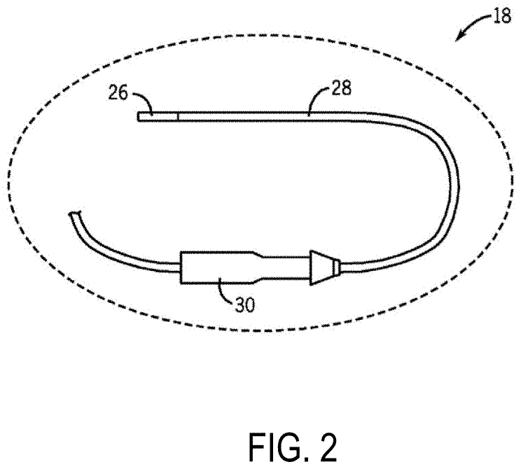

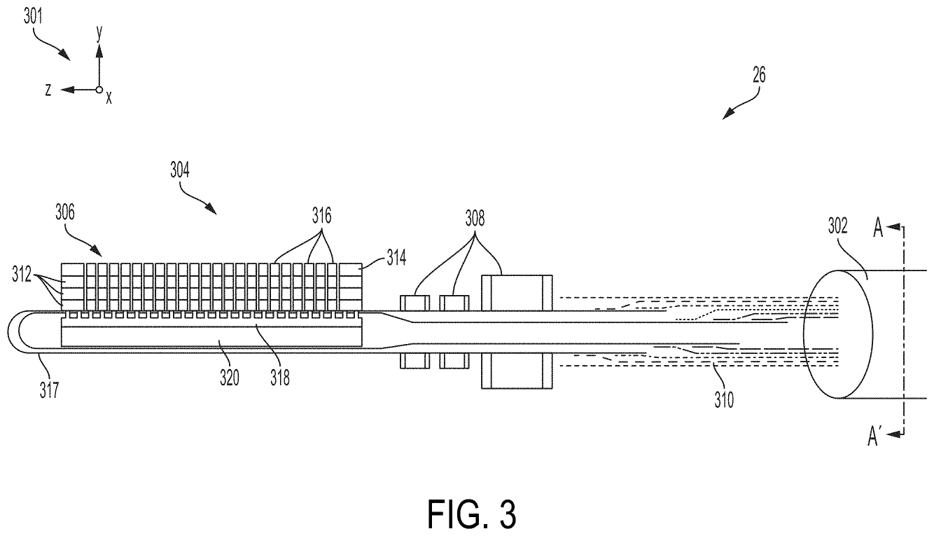

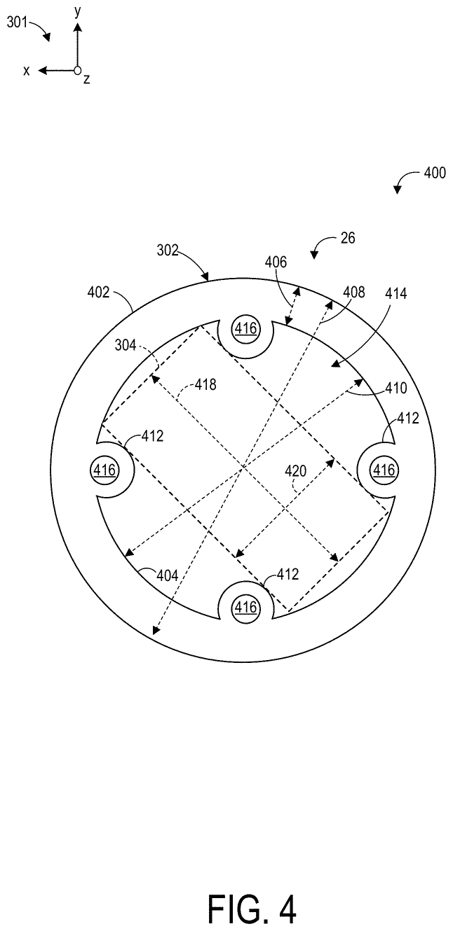

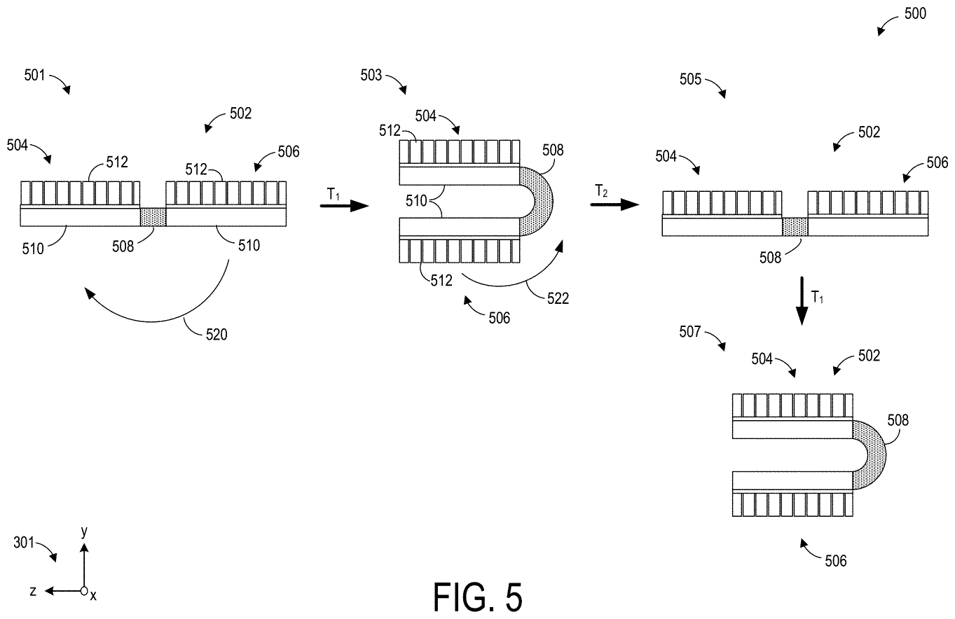

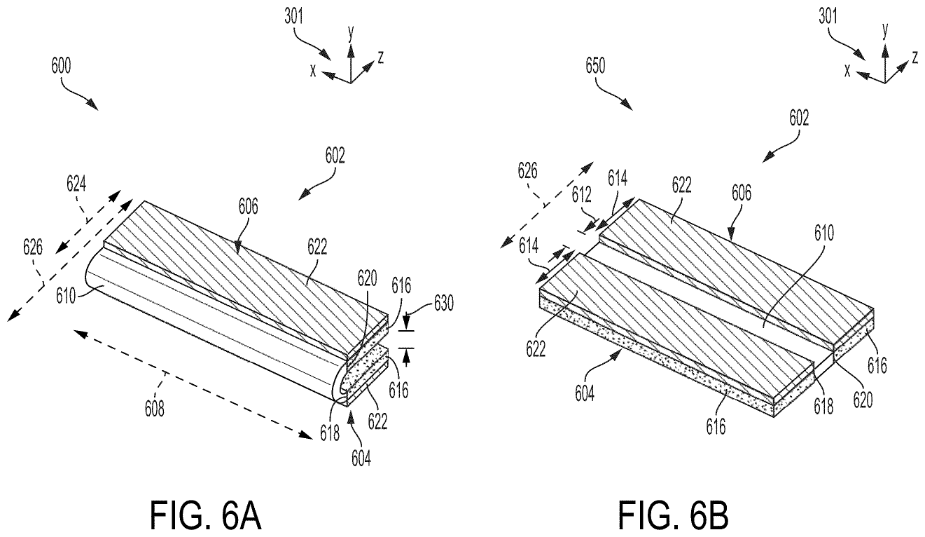

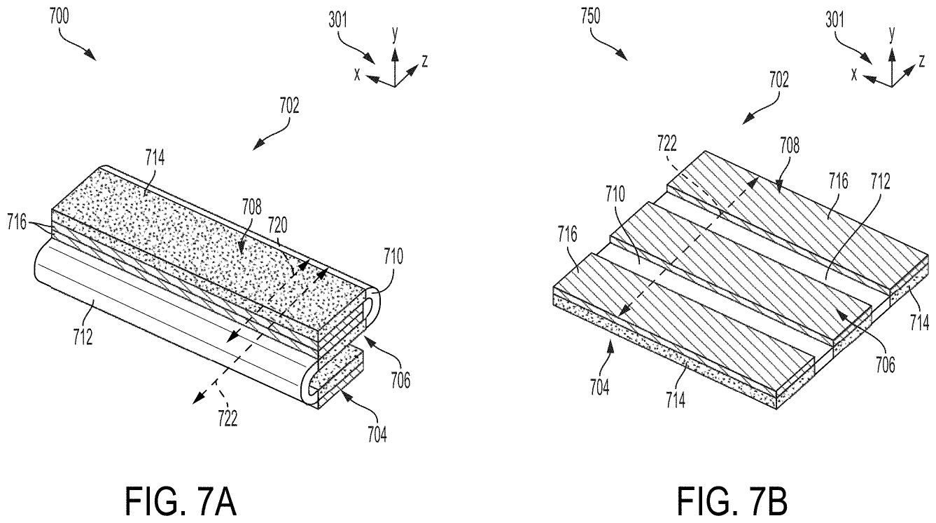

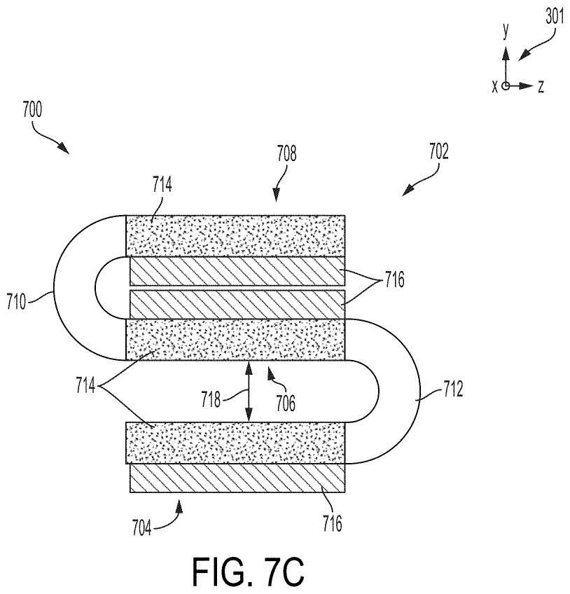

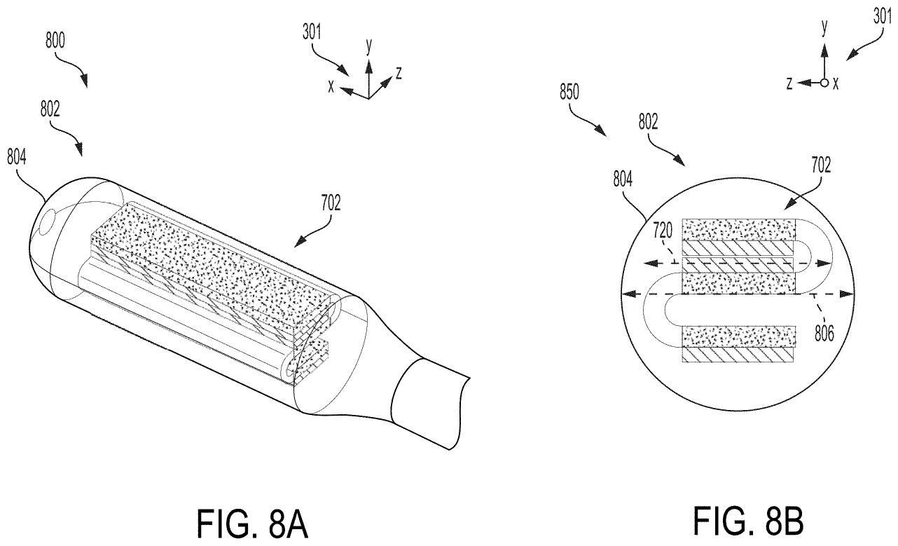

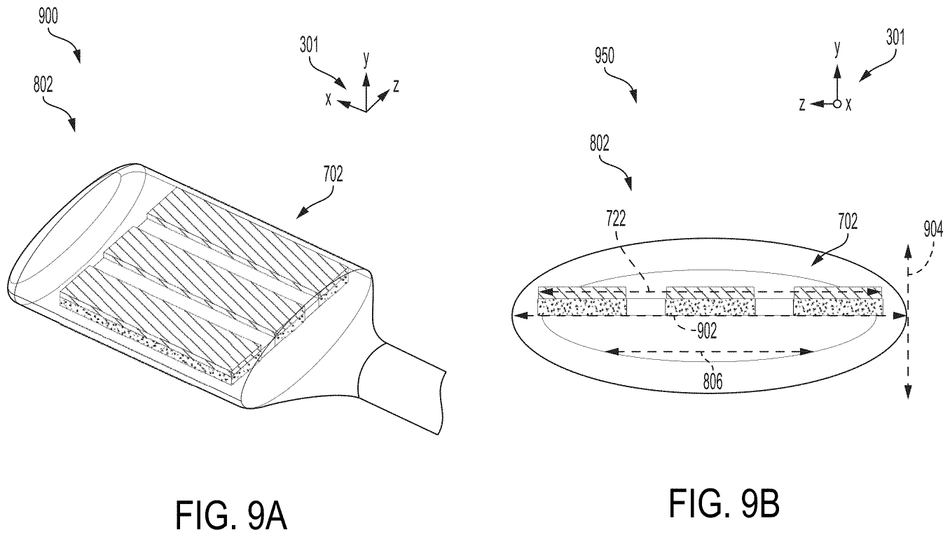

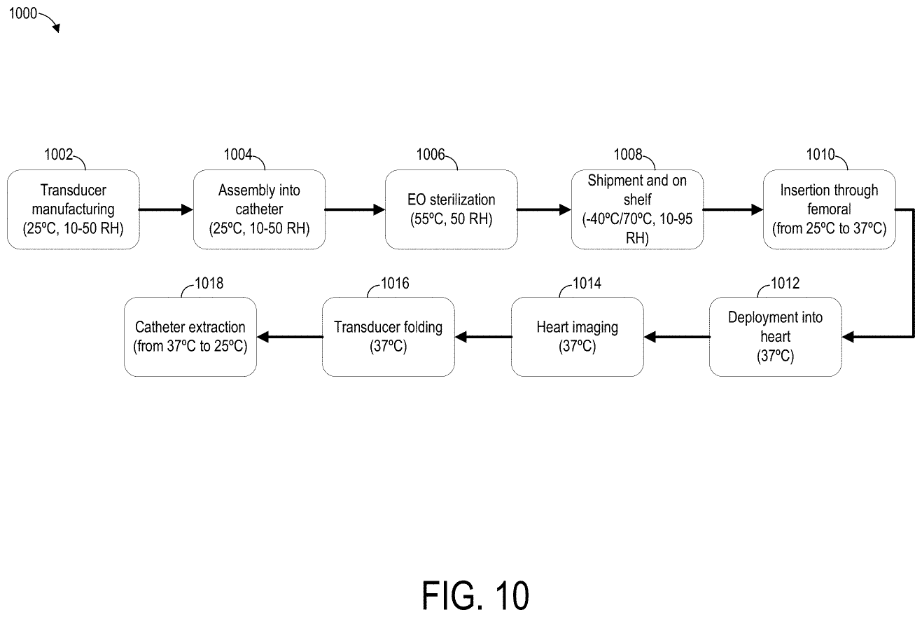

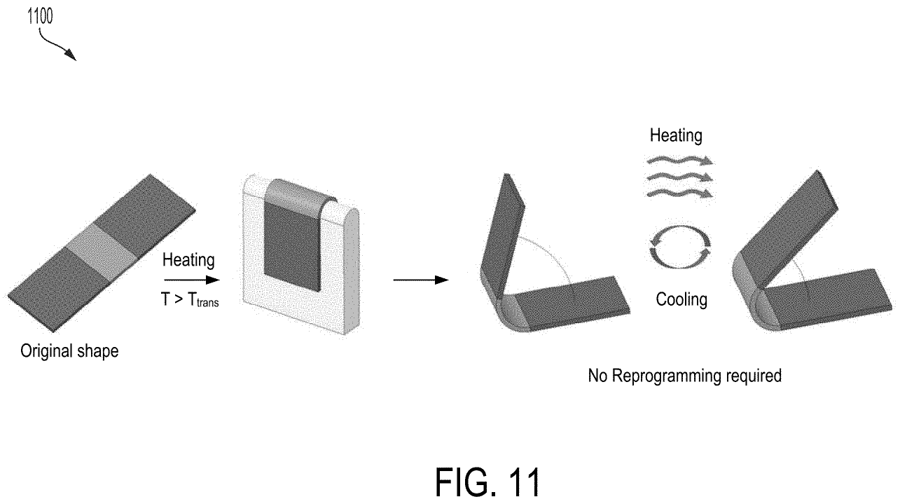

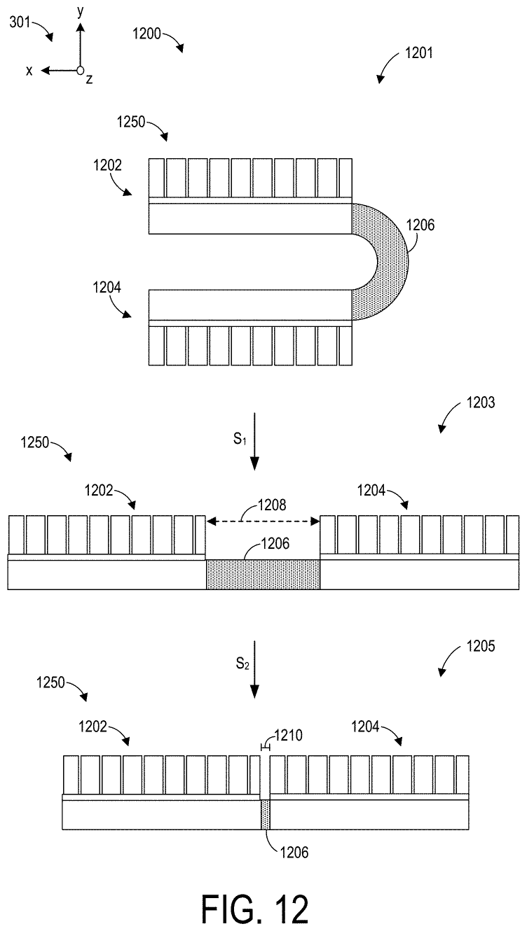

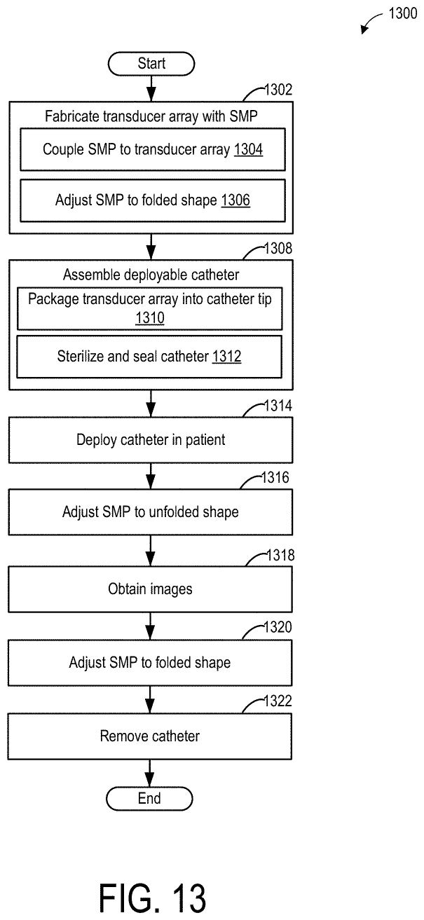

The following description relates to various embodiments of a deployable catheter. The deployable catheter may be included in an imaging system to be inserted into a patient to obtain information about internal tissues and organs. An example of an imaging system equipped with a deployable catheter is shown in FIG. 1. A side view of the deployable catheter is depicted in FIG. 2 and inner components of the deployable catheter are illustrated in a first cross-sectional view of the deployable catheter in FIG. 3. A second cross-sectional view of the deployable catheter is shown as a schematic in FIG. 4. Transitioning of a transducer adapted with a SMP, which may be included in the deployable catheter, between a first shape and a second shape is shown in FIG. 5. Examples of the transducer in the first shape and in the second shape are illustrated in FIGS. 6A-7C. In some examples, the transducer may be enclosed within an inflatable balloon when installed in the deployable catheter. An example of the transducer enclosed in the balloon is depicted in FIGS. 8A-8B when the transducer is in the first shape and in FIGS. 9A-9B when the transducer is in the second shape. A flow diagram outlining a process for assembling and packaging the transducer in the deployable catheter and subsequent deployment of the catheter in a patient is shown in FIG. 10. The SMP may be a two-way shape memory material programmed to alternate between at least two shapes, as shown in FIG. 11. In one example, the SMP may be configured to change shape via more than one type of transition, as illustrated in FIG. 12. An example of a method for assembling and deploying a deployable catheter adapted with a SMP for acquiring intravenous images is shown in FIG. 13.

FIGS. 1-9B and 11-12 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

Medical imaging techniques, such as ultrasound imaging, may be used to obtain real-time data about a patient's tissues, organs, blood flow, etc. However, high resolution data for inner cavities of the tissues and organs may be difficult to obtain via external scanning of the patient. In such instances, a deployable catheter outfitted with a probe may be inserted intravenously into the patient and directed to a target site. The deployable catheter may travel through a narrow channel, such as a vein or artery and therefore may have a similar diameter. However, the narrow diameter of the deployable catheter may limit a size of the probe which, in turn, may constrain data quality NS acquisition speed provided by the probe. For example, when the probe is an ultrasound probe, a resolution and penetration of the ultrasound probe may be determined by a size of a transducer of the probe and in order to increase image quality of the ultrasound probe, a larger transducer than can be enclosed within a housing of the deployable catheter may be demanded.

In one example, the issues described above may be at least partially addressed by incorporating a shape memory material into the deployable catheter. The shape memory material may be a shape memory polymer (SMP) configured to alternate between at least two different shapes. A footprint of a transducer of the deployable catheter, where the SMP is coupled to the transducer, may be selectively increased or decreased. The shape-changing behavior of the SMP allows the transducer to have, for example, a first shape with a first set of dimensions enabling the transducer to be readily inserted into the patient's body within the deployable catheter housing. In response to exposure to a stimulus, the SMP may adjust to a second shape with a second set of dimensions that increases a size of the transducer. By subjecting the SMP to a second stimulus, the SMP may be returned to the first shape, thereby decreasing the size of the transducer. In this way, the imaging probe may be maintained small and easily maneuverable within the patient and enlarged when deployed in a target anatomical region to obtain high resolution data. By leveraging the SMP to induce shape transitions, a cost of the deployable catheter may be maintained low while allowing for a large range of deformation.

Turning now to FIG. 1, a block diagram of an exemplary system 10 for use in medical imaging is illustrated. It will be appreciated that while described as an ultrasound imaging system herein, the system 10 is a non-limiting example of an imaging system which may utilize a deployable device to obtain medical images. Other examples may include incorporating other types of invasive probes such as endoscopes, laparoscopes, surgical probes, intracavity probes, amongst others. The system 10 may be configured to facilitate acquisition of ultrasound image data from a patient 12 via an imaging catheter 14. For example, the imaging catheter 14 may be configured to acquire ultrasound image data representative of a region of interest in the patient 12 such as the cardiac or pulmonary region. In one example, the imaging catheter 14 may be configured to function as an invasive probe. Reference numeral 16 is representative of a portion of the imaging catheter 14 disposed inside the patient 12, such as inserted into a vein. Reference numeral 18 is indicative of a portion of the imaging catheter 14 depicted in greater detail in FIG. 2.

The system 10 may also include an ultrasound imaging system 20 that is in operative association with the imaging catheter 14 and configured to facilitate acquisition of ultrasound image data. It should be noted that although the exemplary embodiments illustrated hereinafter are described in the context of a medical imaging system, such as an ultrasound imaging system, other imaging systems and applications are also contemplated (e.g., industrial applications, such as nondestructive testing, borescopes, and other applications where ultrasound imaging within confined spaces may be used). Further, the ultrasound imaging system 20 may be configured to display an image representative of a current position of the imaging catheter tip within the patient 12. As illustrated in FIG. 1, the ultrasound imaging system 20 may include a display area 22 and a user interface area 24. In some examples, the display area 22 of the ultrasound imaging system 20 may be configured to display a two- or three-dimensional image generated by the ultrasound imaging system 20 based on the image data acquired via the imaging catheter 14. For example, the display area 22 may be a suitable CRT or LCD display on which ultrasound images may be viewed. The user interface area 24 may include an operator interface device configured to aid the operator in identifying a region of interest to be imaged. The operator interface may include a keyboard, mouse, trackball, joystick, touch screen, or any other suitable interface device.

FIG. 2 illustrates an enlarged view of the portion 18 shown in FIG. 1 of the imaging catheter 14. As depicted in FIG. 2, the imaging catheter 14 may include a tip 26 on a distal end of a flexible shaft 28. The catheter tip 26 may house a transducer and motor assembly. The transducer may include one or more transducer arrays, each transducer array including one or more transducer elements. The imaging catheter 14 may also include a handle 30 configured to facilitate an operator manipulating the flexible shaft 28.

An example of the catheter tip 26 of FIG. 2 is shown in FIG. 3. A set of reference axes 301 are provided, indicating a y-axis, an x-axis, and a z-axis. The catheter tip 26 may have a housing 302 surrounding a transducer 304 which may include at least one transducer array 306, capacitors 308, and a catheter cable 310. The other components not shown in FIG. 3 may also be enclosed within the housing 302, such as a motor, a motor holder, a thermistor, and an optional lens, for example. Furthermore, in some examples, the catheter tip 26 may include a system for filling the tip with a fluid, such as an acoustic coupling fluid.

The transducer array 306 has several layers stacked along the y-axis and extending along the x-z plane. One or more layers of the transducer array 306 may be layers of transducer elements 312. In one example, the transducer elements 312 may be piezoelectric elements, where each piezoelectric element may be a block formed of a natural material such as quartz, or a synthetic material, such as lead zirconate titanate, that deforms and vibrates when a voltage is applied by, for example, a transmitter. In some examples, the piezoelectric element may be a single crystal with crystallographic axes, such as lithium niobate and PMN-PT (Pb(Mg_{1/3}Nb_{2/3})O₃—PbTiO₃). The vibration of the piezoelectric element generates an ultrasonic signal formed of ultrasonic waves that are transmitted out of the catheter tip 26. The piezoelectric element may also receive ultrasonic waves, such as ultrasonic waves reflected from a target object, and convert the ultrasonic waves to a voltage. The voltage may be transmitted to a receiver of the imaging system and processed into an image.

An acoustic matching layer 314 may be positioned above the transducer elements 312. The acoustic matching layer 314 may be a material positioned between the transducer elements 312 and a target object to be imaged. By arranging the acoustic matching layer 314 in between, the ultrasonic waves may first pass through the acoustic matching layer 314, and emerge from the acoustic matching layer 314 in phase, thereby reducing a likelihood of reflection at the target object. The acoustic matching layer 314 may shorten a pulse length of the ultrasonic signal, thereby increasing an axial resolution of the signal.

The layers formed by the acoustic matching layer 314 and the transducer elements 312 may be diced along at least one of the y-x plane and the y-z plane to form individual acoustic stacks 316. Each of the acoustic stacks 316 may be electrically insulated from adjacent acoustic stacks but may all be coupled to at least one common layer positioned below or above the transducer elements, with respect to the y-axis.

An electrical circuit 318 may be layered below, relative to the y-axis, the transducer elements 312. In one example, the electrical circuit may be at least one application-specific integrated circuit (ASIC) 318 directly in contact with each of the acoustic stacks 316. Each ASIC 318 may be coupled to one or more flex circuits 317 which may extend continuously between the transducer array 306 and the catheter cable 310. The flex circuits 317 may be electrically coupled to the catheter cable 310 to enable transmission of electrical signals between the transducer array 306 and an imaging system, e.g., the imaging system 20 of FIG. 1. The electrical signals may be tuned by the capacitors 308 during transmission.

An acoustic backing layer 320 may be arranged below the ASIC 318, with respect to the z-axis. In some examples, as shown in FIG. 3, the backing layer 320 may be a continuous layer of material that extends along the x-z plane. The backing layer 320 may be configured to absorb and attenuate backscattered waves from the transducer elements 312. A bandwidth of an acoustic signal generated by the transducer elements 312, as well as the axial resolution, may be increased by the backing 126.

As described above, the transducer 30, the capacitors 308, and the catheter cable 310 may be enclosed within the housing 302. Thus a size, e.g., a diameter or width of the components may be determined by an inner diameter of the housing 302. An inner diameter of the housing 302 may be, in turn, determined by an outer diameter and a desirable thickness of the housing 302. The outer diameter of the housing 302 may be constrained by a region of a patient's body through which the imaging catheter is inserted. For example, the imaging catheter may be an intracardiac echocardiography (ICE) catheter used to obtain images of cardiac structures and blood flow inside the patient's heart.

The imaging catheter may be introduced into the heart through the aorta, inferior vena cava, or jugular vein. In some instances, the imaging catheter may be fed through regions with narrower diameters, such as the coronary sinus, the tricuspid valve, and the pulmonary artery. As such, the outer diameter of the imaging catheter may not be greater than 10 Fr or 3.33 mm. The outer diameter and corresponding inner diameter of the imaging catheter housing are shown in FIG. 4 in a cross-section 400 of the housing 302 of the catheter tip 26, taken along line A-A′ depicted in FIG. 3.

As shown in FIG. 4, an outer surface 402 of the housing 302 of the imaging catheter may be spaced away from an inner surface 404 of the housing 302 by a thickness 406 of the housing 302. The thickness 406 of the housing 302 may be optimized to provide the housing 302 with a target degree of structural stability, e.g. resistance to deformation, balanced with flexibility, e.g., ability to bend when a force is applied. In one example, an outer diameter 408 of the housing 302 may be 3.33 mm, the thickness 406 may be 0.71 mm, and an inner diameter 410 of the housing 302 may be 2.62 mm. In other examples, the outer diameter of the housing may be between 2-5 mm, the thickness may be between 0.24-1 mm, and the inner diameter may be between 1-4 mm. In yet other examples, the imaging catheter may have a variety of dimensions, depending on application. For example, an endoscope may have an outer diameter 10-12 mm. It will be appreciated that the imaging catheter may have various diameters and sizes without departing from the scope of the present disclosure.

The inner surface 404 of the housing 302 may include lobes 412 protruding into an inner volume, or lumen 414 of the housing 302. The lobes 412 may be semi-circular projections, each enclosing an individual lumen 416 for housing a steering wire of the imaging catheter. An arrangement of the transducer 304 of the imaging catheter within the lumen 414 of the housing 302 is indicated by a dashed rectangle. A maximum elevation aperture 418 of the transducer 304 may be determined based on the inner diameter 410 of the housing 302 and a height 420 of the transducer 304 may be configured to fit between the lobes 412 of the housing 302. In one example, the elevation aperture 418 may be a maximum of 2.5 mm and the height 420 may be a maximum of 1 mm.

As described above, dimensions of the transducer 304 may be determined by the inner diameter 410, thickness 406, and outer diameter 408 of the housing 302 which may, in turn, be determined based on insertion of the imaging catheter into specific regions of the patient's anatomy. The constraints imposed on a size of the transducer 304 and diameter 422 of the catheter cable, may affect a resolution, penetration, and fabrication of the transducer 304. Each of the resolution, penetration and ease of fabrication may be enhanced by increasing the size of the transducer 304 but the geometry of the transducer 304, and therefore performance, is bound by the dimensions of the catheter housing 302 in order for the deployable catheter to travel intravenously through a patient.

In one example, the transducer may be enlarged upon deployment at a target site by adapting the transducer with a shape memory material. The shape memory material may be a shape memory polymer (SMP) configured to respond mechanically to one or more stimuli. Examples of SMPs include linear block copolymers, such as polyurethanes, polyethylene terephthalate, polyethyleneoxide, and other thermoplastic polymers such as polynorbornene. In one example, the SMP may be a powder mixture of silicone and tungsten in an acrylic resin. The SMP may be stimulated by physical stimuli, such as temperature, moisture, light, magnetic energy, electricity, etc., by chemical stimuli, such as chemicals, pH level, etc., and by biological stimuli, such as presence of glucose and enzymes. When applied to an imaging catheter, the transducer may incorporate the SMP to enable a shape of the transducer to be altered upon exposure to at least one stimulus. The SMP may have physical properties as provided below in Table 1 which may offer more desirable characteristics than other types of shape memory materials, such as shape memory alloys. For example, SMPs may have a higher capacity for elastic deformation, lower cost, lower density, as well as greater biocompatibility and biodegradability. In particular, the lower cost of SMPs may be desirable for application in disposable deployable catheters.

TABLE 1
Physical Properties of Shape Memory Polymers

| Property | Range |
|---|---|
| Density (g/cm³) | 0.2-3 |
| Extent of deformation | Up to 800% |
| Required stress for deformation (MPa) | 1-3 |
| Stress generated upon recovery (MPa) | 1-3 |
| Transition temperature (° C.) | −10 to 100 |
| Recovery speed | 1 s to 1 hr |
| Processing condition | <200° C.; low pressure |
| Cost | <$10/lb |

In one example, the SMP may have two-way shape memory so that the SMP may adjust between two shapes without demanding reprogramming or application of an external force. For example, the SMP may convert to a temporary shape in response to a first stimulus and revert to a permanent shape in response to a second stimulus. The first and second stimuli may be of a same or different type, e.g., the first stimulus may be a high temperature and the second stimulus may be a low temperature or the first stimulus may be a humidity level and the second stimulus may be a threshold temperature. The two-way shape memory behavior is neither mechanically nor structurally constrained, thereby allowing the SMP to switch between the temporary shape and permanent shape without applying the external force.

As an example, conversion of a transducer 502 between a first shape and a second shape is shown in a first diagram 500 in FIG. 5. The transducer 502 includes a first transducer array 504 and a second transducer array 506 where the second transducer array 506 is aligned with the first transducer array 504 along the z-axis and spaced away from the first transducer array 504. In other words, the transducer 502 has an overall planar shape with the first and second transducer arrays 504, 506 co-planar with one another along a common plane, e.g., the x-z plane. A first step 501 of the first diagram 500, depicts coupling of an SMP 508 to a backing layer 510 of each of the first and second transducer arrays 504, 506. The SMP 508, configured as a two-way memory SMP, is arranged between the transducer arrays along the z-axis and may be fixedly attached to edges of the backing layers 510 and arranged co-planar with the backing layers 510. For example, the backing layers 510 and the SMP 508 arranged therebetween may form a continuous, planar unit. Transducer elements 512 are laminated onto the backing layer 510 of the first and second transducer arrays 504, 506.

In some examples, the SMP 508 may form a continuous layer entirely across the transducer 502. The SMP 508 may form an acoustic layer of the transducer 502, such as a matching layer or a backing layer. By incorporating the SMP 508 as an acoustic layer, an assembly and number of components of the transducer array may be simplified without adversely affecting a reduction in the transducer array footprint.

The transducer 502 is exposed to a first temperature, T₁, and, at a second step 503, the SMP 508 changes shape in response to T₁. The SMP 508 may bend into a semi-circular shape, pivoting the second transducer array 506 substantially through 180 degrees along a first rotational direction, e.g., clockwise, as indicated by arrow 520. Bending, as referred to herein, may be any transitioning of a planar structure to a non-planar conformation. As such, various deformations of the structure from a configuration that is aligned with a plane may be considered bending.

When the SMP 508 bends, the transducer 502 may therefore also bend. While the SMP may bend through a range of angles, bending of the SMP so that two regions of the transducer 502 become stacked over one another and substantially parallel with one another is referred to as folding herein. The SMP, in some examples, may not bend to an extent that the transducer is folded. However, folding of the transducer may provide a most compact conformation of the transducer to enable passage of the deployable catheter through intravenous passages.

As a result of the folding of the transducer 502, the second transducer array 506 is positioned under the first transducer array 504, with respect to the y-axis, in a folded shape. An overall surface area of the transducer elements 512, including the transducer elements 512 of both the first and second transducer arrays 504, 506, is reduced at the second step 503 compared to the first step 501 when viewing the transducer 502 along the y-axis.

The transducer 502 is exposed to a second temperature, T₂, and, in response, the SWP 508 reverts to the planar geometry of the first step 501 at a third step 505 of the first diagram 500. The second transducer array 506 is pivoted substantially through 180 degrees along a second rotational direction, opposite of the first rotational direction, e.g., counterclockwise. The second temperature T₂ may be a higher or lower temperature than T₁. Subjecting the transducer 502 to T₁ again compels the SMP 508 to bend, folding the transducer 502 so that the second transducer array 506 is pivoted 180 degrees at a fourth step 507.

The steps shown in the first diagram 500 may be repeated many times. For example, prior to insertion of an imaging catheter adapted with the transducer 502 of FIG. 5 in to a patient, the transducer 502 may be initially exposed to T₁ to fold and decrease the size of the transducer 502. The folded transducer 502, may fit within a housing of the imaging catheter and inserted intravenously into the patient. When the transducer 502 reaches a target site within the patient, the transducer 502 may be unfolded and enlarged by subjecting the array to T₂. Images may be obtained while the transducer 502 is unfolded and increased in size. For example, unfolding the transducer 502 may increase an elevation aperture of the transducer 502.

When scanning is complete, the transducer 502 may be exposed again to T₁ to cause the transducer 502 to fold and decrease in size. The imaging catheter may then be withdrawn from the site and removed from the patient or deployed to another site for imaging within the patient. Thus the shape and size of the transducer 502 may be adjusted between the planar and folded configurations numerous times during an imaging session.

It will be appreciated that the configurations of the transducer 502 shown in FIG. 5 are non-limiting examples of shapes that the transducer may transition between. Other examples may include the transducer 502 being in a non-planar geometry at the first step 501, such as slightly bent shape, becoming more bent at the second step 503, and alternating between the less bent and more bent shapes upon exposure to one or more stimuli. In addition, the transducer 502 may fold so that the first and second transducer arrays 504, 506, are not parallel with one another. In yet other examples, the first and second transducer arrays 504, 506 may be different sizes.

Furthermore, when the SMP 508 forms an entire layer across the transducer 502, rather than forming a section between the backing layers 510 of the first and second transducer arrays 504, 506, the SMP 508 may be adapted to change shape only in an area between the transducer arrays. In one example the SMP 508 may be able to change shape via more than one type of transition. For example, the SMP 508 may bend upon exposure to one type of stimulus and shrink upon exposure to another type of stimulus. In another example, the SMP 508 may include more than one type of shape memory material. As an example, the SMP 508 may be formed of a first type of material configured to bend and a second type of material configured to shrink. Other variations in shape transitions, combination of materials, and positioning of the SMP 508 within the transducers have been contemplated.

While temperature changes are described as a stimulus for inducing changes in the SMP shape for the first diagram 500 of FIG. 5, it will be appreciated that the first diagram 500 is a non-limiting example of how deformation of the SMP may be triggered. Other types of stimuli, such as humidity, pH, UV light, etc. may be used to induce mechanical changes in the SMP. More than one type of stimulus may be applied to the SMP to achieve similar or different shape modification. Furthermore, deformation of the SMP may include other manners of shape change other than bending. For example, the SMP may curl into a jellyroll configuration or shrink along at least one dimension. Details of the mechanical deformation and stimuli used to elicit the deformation are described further below.

In some examples, as shown in FIG. 5, a transducer of a deployable catheter may include two sections, or two transducer arrays. Each transducer array may include one or more acoustic stacks, including, as described above with reference to FIG. 2, a matching layer, an element, and a backing layer. An ASIC may be coupled to each transducer array. A first example of a transducer 602 incorporating a SMP to enable modification of an active area of the transducer 602 is shown in FIGS. 6A and 6B. The transducer 602 is shown in a first, folded configuration 600 in FIG. 6A and in a second, unfolded configuration 650 in FIG. 6B.

The transducer 602 has a first transducer array 604 and a second transducer array 606. The first and second transducer arrays 604, 606 have similar dimensions and are each rectangular and longitudinally aligned with the x-axis, e.g., a length 608 of each transducer array is parallel with the x-axis. A SMP 610 is arranged between the transducer arrays, along the z-axis. In other words, the first transducer array 604 is spaced away from the second transducer array 606 by a width 612 of the SMP 610, as shown in FIG. 6B. The width 612 of the SMP 610 may be less than a width 614 of each of the first and second transducer arrays 604, 606 while a length of the SMP 610, defined along the x-axis, may be similar to the length 608 of the transducer arrays.

The SMP 610 may be connected to inner edges of a backing layer 616 of each of the first and second transducer arrays 604, 606. For example, the SMP 610 may be directly in contact with and adhered to a longitudinal inner edge 618 of the backing layer 616 of the first transducer array 604, e.g., an edge of the backing layer 616 facing the second transducer array 606 and aligned with the x-axis, and to a longitudinal inner edge 620 of the backing layer 616 of the second transducer array 606, e.g., an edge of the backing layer 616 facing the first transducer array 604 and aligned with the x-axis. A thickness of the SMP 610 may be similar to a thickness of the backing layer 616 of each of the first and second transducer arrays 604, 606, the thicknesses defined along the y-axis. A matching layer 622 is stacked above the backing layer 616 of each of the transducer arrays. An element, e.g., a piezoelectric element, may be arranged between the matching layer 622 and the backing layer 616 (not shown in FIGS. 6A and 6B).

When in the first configuration 600 as shown in FIG. 6A, the SMP 610 is curved into a semi-circular shape. The second transducer array 606 is stacked directly over, with respect to the y-axis, and spaced away from the first transducer array 604, so that both transducers are maintained co-planar with the x-z plane. The transducer 602 is folded in FIG. 6A so that each matching layer 622 of the transducer arrays face away outwards and away from one another and the backing layers 616 of the transducer arrays face one another. The backing layers 616 may be spaced away from one another by a distance 630 similar to a diameter of the semi-circle formed by the SMP 610. However, in other examples, the transducer 602 may be folded in an opposite direction so that the backing layers 616 of the transducer arrays face one another and the matching layers 622 face away from one another.

As the transducer 602 transitions between the first and second configurations 600, 650, at least one of the transducer arrays are pivoted, for example, 180 degrees relative to the other transducer array. For example, when adjusting from the first configuration 600 to the second configuration 650, the first transducer array 604 may be pivoted through a first rotational direction to become co-planar with the second transducer array 606. Alternatively, the second transducer array 606 may be pivoted 180 degrees through a second rotational direction, opposite of the first rotational direction. The first transducer array 604 may be pivoted through the second rotational direction or the second transducer array 606 may be pivoted through the first rotational direction to return the transducer 602 to the first configuration 600. In another example, both transducer arrays may be pivoted through 90 degrees to achieve transitioning between the first and second configurations 600, 650. It will be appreciated that description of the pivoting of the transducer arrays through 180 degrees is for illustrative purposes and other examples may include the transducer arrays pivoting more or less than 180 degrees.

In the first configuration 600, a width 624 of the transducer 602 is reduced relative to a width 626 of the transducer 602 in the second configuration 650. An active area of the transducer 602 may be equal to a surface area of one of the first or second transducer arrays 604, 606. In the second configuration 650, with the first and second transducer arrays 604, 606 co-planar with one another and side-by-side, the active area of the transducer 602 is doubled relative to the first configuration 600. As such, an elevation aperture of the transducer 602 is at least doubled when unfolded into the second configuration 650, thereby increasing a resolution and penetration of the transducer 602.

In another example, a transducer of an imaging probe may include more than two sections or transducer arrays. A second example of a transducer 702 is shown in a first, folded configuration 700 in FIGS. 7A and 7C, and a second, unfolded configuration 750 in FIG. 7B. The transducer 702 includes a first transducer array 704, a second transducer array 706, and a third transducer array 708. All three transducer arrays may have similar dimensions and geometries and may be connected by a first SMP 710 and a second SMP 712.

For example, the transducer arrays may be spaced away from one another but co-planar and aligned along the x-axis and z-axis in the second configuration 750 of FIG. 7B. The first transducer array 704 is spaced away from the second transducer array 706 by the first SMP 710 and the second transducer array 706 is spaced away from the third transducer array 708 by the second SMP 712. As described above for the first example of the transducer 602 of FIGS. 6A-6B, the SMPs may be directly connected to longitudinal inner edges of the transducer arrays along a backing layer 714 of each transducer array. The SMPs may be co-planar and have a similar thickness to the backing layer 714 of the transducer arrays. A matching layer 716 of each of the transducer arrays is positioned above the backing layer 714 and aligned with each backing layer 714 along the y-axis. As such, the matching layer 716 protrudes above the first and second SMPs 710, 712 with respect to the y-axis. An element may be arranged between the matching layer 716 and the backing layer 714 (not shown in FIGS. 7A and 7B).

In the first configuration 700 of FIG. 7A, the transducer 702 is folded into an S-shaped geometry when viewed along the x-axis, as shown in FIG. 7C. In the S-shaped geometry, the first SMP 710 is bent into a semi-circle, forming a right half of a circle. The first transducer array 704 may be pivoted through a first rotational direction relative to the second transducer array 706 so that the second transducer array 706 is stacked over and aligned with the first transducer array 704 with respect to the y-axis. While the backing layer 714 of the second transducer array 706 and the backing layer 714 of the first transducer array 704 face each other with no other component of the transducer 702 positioned therebetween, the backing layer 714 of the transducer arrays are spaced apart by a distance 718 similar to a diameter of the semi-circle formed by the first SMP 710.

The second SMP 712 is bent in an opposite direction from the first SMP 710, into a semi-circle forming a left half of a circle. The bending of the second SMP 712 causes the third transducer array 708 to be stacked over the second transducer array 706 along the y-axis. The third transducer array 708 is pivoted through a second rotational direction, opposite of the first rotation direction, so that the third transducer array 708 is aligned with both the first and second transducer arrays 704, 706, along the y-axis and the matching layer 716 of the third transducer array 708 faces the matching layer 716 of the second transducer array 706. The matching layers 716 of the second and third transducer arrays 706, 708 are separated by a gap that is smaller than the distance 718 between the backing layers 714 of the first and second transducer arrays 704, 706.

As the transducer 702 transitions between the first and second configurations 700, 750, at the first and third transducer arrays 704, 708, may be pivoted through 180 degrees in opposite rotation directions, relative to the second transducer array 706. For example, when adjusting from the first configuration 700 to the second configuration 750, the first transducer array 704 may be pivoted through a first rotational direction to become co-planar with the second transducer array 606. The third transducer array 708 may be pivoted through a second rotational direction, opposite of the first rotational direction to also become co-planar with the second transducer array 606. To return the transducer 702 to the first configuration 700 from the second configuration 750, the first transducer array 704 may be pivoted 180 degrees through the second rotational direction and the second transducer array 706 may be pivoted 180 degrees through the first rotational direction. Alternatively, on other examples, the transducer arrays may be pivoted opposite of the transitioning described above. It will be appreciated that description of the pivoting of the transducer arrays through 180 degrees is for illustrative purposes and other examples may include the transducer arrays pivoting through more or less than 180 degrees.

A width 720, as shown in FIG. 7A, of the transducer 702 in the first configuration 700 may be narrower than a width 722 of the transducer 702 in the second configuration 750. An active area of the transducer 702, determined by a total transducer array surface area along the x-z plane, may be increased threefold when the transducer 702 is adjusted from the first configuration 700 to the second configuration 750. Thus, when a transducer is formed of three transducer arrays (a 3-section transducer, hereafter), and the unfolded 3-section transducer, e.g., the second configuration 750 of FIG. 7B, is equal in size to an unfolded transducer with two transducer arrays (a 2-section transducer, hereafter), e.g., the second configuration 650 of FIG. 6B, the transducer arrays of the 3-section transducer may be narrower in width than the transducer arrays of the 2-section transducer. When folded, the 3-section transducer may have a smaller footprint than the 2-section transducer and may thereby be inserted through narrower channels.

Alternatively, the transducer arrays of the 3-section and 2-section transducers may be similar in size. When folded, both the transducers may have a similar footprint. However, when deployed and unfolded in a target scanning site, the 3-section transducer may have a larger active area, allowing the 3-section transducer to have greater resolution and penetration than the 2-section transducer. Furthermore, the first and second examples of the transducer shown in FIGS. 6A-7C are non-limiting examples. Other examples may include transducers with more than three sections, or transducers and transducer arrays with different geometries and dimensions from those shown.

The folding of a transducer compelled by an SMP, as illustrated in FIGS. 5-7C, may be leveraged to allow the transducer to be implemented in a deployable catheter, such as the imaging catheter 14 of FIG. 1, without inhibiting passage of the deployable catheter through narrow arteries and veins. For example, as shown in a perspective view 800 in FIG. 8A and an end view 850 in FIG. 8B, the transducer 702 of FIGS. 7A-7C may be employed in a catheter tip 802. In one example, the catheter tip 802 may be the catheter tip 26 of FIGS. 2-4.

The catheter tip 802 may be a tip of a balloon catheter, having a balloon 804 at a terminal end of the catheter tip 802. The balloon 804 may be a compartment formed of a thin, flexible material, inflatable material, such as polyester, polyurethane, silicone, etc. The balloon 804 may be used to increase a size of a region in which the catheter tip 802 is deployed by inflating the balloon 804.

The transducer 702 is placed entirely inside of the balloon 804. In FIGS. 8A-8B, the balloon 804 is not inflated and the transducer 702 is in the first, folded configuration (e.g., as shown in FIGS. 7A and 7C). The balloon 804 may be substantially cylindrical, as shown in FIG. 8A, with an inner diameter 806 that is wider than the width 720 of the folded transducer 702, as shown in FIG. 8B.

The balloon 804 may be inflated, as shown in a perspective view 900 in FIG. 9A and an end view 950 in FIG. 9B of the catheter tip 802. When inflated, the balloon 804 may be configured to expand mostly along one axis, such as the along the z-axis, resulting in an elliptical geometry of balloon 804 when viewed along the x-axis, as shown in FIG. 9B. For example, a width 902 of the balloon 804 may be greater than the diameter 806 of the balloon 804 when the balloon 804 is not inflated while a height 904 of the balloon 804 may become smaller than or remain similar to the diameter 806 of the balloon 804 when the balloon is not inflated.

The balloon 804 may be inflated by adding a fluid to the balloon 804. For example, a liquid, such as water or a saline solution may be added to the balloon 804 to increase a volume of the balloon 804 to a target volume that accommodates a size of the transducer 702 when the transducer 702 is unfolded, as shown in FIGS. 9A and 9B. In other examples, a gas may be used to expand the balloon 804, such as air or nitrogen.

When the balloon 804 is inflated, the transducer 702 may be adjusted to the second, unfolded configuration. The width 902 of the inflated balloon 804 may be wider than the width 722 of the unfolded transducer 702, allowing the transducer 702 to unfold without inhibition to obtain imaging data at a target site. The material of the balloon 804, as well as the fluid used to inflate the balloon 804, may be selected based on a lack of interference of the material and fluid on transmission of imaging signals between the transducer 702 and the target site. For example, when the transducer 702 is implemented in an ultrasound probe, the balloon material and fluid do not attenuate or absorb at ultrasonic frequencies.

As an example, when the transducer is in the first, folded configuration, as shown in FIGS. 8A and 8B, and enclosed in the uninflated balloon, the SMPs of the transducer may be in a first, permanent shape when the SMPs are two-way memory shape polymers. The transducer may remain in the first shape while under a first condition, such as temperature, humidity, pH, etc., until the catheter tip reaches the target site and the balloon is inflated.

Once inflated, the transducer may be exposed to a second condition which triggers a shape change of the SMPs to the second, unfolded configuration. The second conditions may be maintained until scanning and data acquisition by the transducer is complete. The transducer may then be subjected to the first condition to return the transducer to the first, folded configuration. The balloon may be deflated by draining/venting the balloon.

In the first configuration of the transducer, the narrower diameter of the catheter tip, compared to the second configuration, may allow the catheter tip to be readily inserted through narrow pathways in the patient's body. The active area may be expanded to increase a capability and data quality of the transducer when the catheter tip is deployed at the target site and the balloon is inflated. The catheter tip may then be withdrawn from the target site by inducing the transducer to convert to the first configuration and deflating the balloon.

The change in footprint of the active area of the transducer between the first and second configurations is enabled by the folding of the transducer. Folding of the transducer at regions between the transducer arrays allows a coupling of rigid ASICs to each transducer array to be maintained while varying the size of the active area. The folded configurations shown in FIGS. 5, 6A, 7A, 7C, and 8A-8B show a pivoting of at least one transducer array by 180 degrees relative to an adjacent, stationary transducer array. It will be appreciated that such a description is for illustrative purposes and in other examples, each transducer array may be pivoted during transitioning between shapes. Furthermore, in other examples, the transducer array may be pivoted through different ranges of angles. For example, at least one transducer array may be pivoted 90 degrees, 120 degrees, or any angle between 0 to 360 degrees relative to the adjacent, stationary transducer array.

An SMP of a transducer may be configured to respond rapidly with high sensitivity to stimuli. For example, as shown in FIG. 10, a flow diagram 1000 illustrates physical conditions the transducer may be subjected to during fabrication, relocation, and subsequent deployment into a patient's heart. At step 1002, the transducer, incorporating one or more transducer arrays with a SMP, may be manufactured at room temperature and at ambient humidity of 10-50%. The transducer may be programmed to be in either a folded or unfolded configuration during fabrication. The transducer is then induced to fold, if manufactured in the unfolded configuration, to decrease a size of the transducer, and then assembled into a deployable catheter at step 1004 under similar temperature and humidity. The catheter is sterilized with ethylene oxide at step 1006 at which the catheter is exposed to a higher temperature of 55° C. and 50% relative humidity. The sterilized catheter is then shipped to a facility at 1008, such as a warehouse, for shelf storage. During transport and storage, the catheter may be subject to a wide temperature range, such as between −40-70° C. and 10-95% relative humidity.

In order to circumvent unfolding of the catheter during transport and storage, the SMP may be configured to respond only when the temperatures rise above, for example, 70° C. Alternatively, the SMP may be formed of a material that responds to a chemical stimulus and may exposure to the chemical stimulus during transport and storage is unlikely. Furthermore, in another example, the shape of the catheter may be maintained using mechanical constraints.

The catheter may be selected to perform cardiac imaging. The catheter may be coupled to an imaging system, such as the imaging system 10 of FIG. 1, and inserted through a femoral artery at step 1010. The temperature experienced by the catheter during insertion and passage to the heart may increase from 25° C. to 37° C. The relative humidity, however, may not vary significantly due to sealing of the transducer within a balloon. The catheter is deployed into the heart at step 1012 and unfolded. An active area of the transducer is increased and images are obtained at step 1014. During these steps, the temperature remains constant at 37° C. At step 1016 the temperature is still 37° C. but folding of the transducer is induced. The folding may be activated by a different stimuli other than temperature. For example, humidity may be modified or visible light, ultraviolet light may be varied or magnetic energy or electricity may be applied. The catheter is extracted at step 1018 and subjected to a decrease in temperature from 37° C. to 25° C.

In order for the deployable catheter to be used to image a target anatomical region, such as the heart, by insertion through an artery or vein, to obtain high resolution images in real-time, deployment of the catheter may be configured to adhere to stringent parameters. For example, the transition of the SMP of the transducer between folded and unfolded configurations may occur within one minute or less. For example, the SMP may convert from the folded to unfolded configuration within 20 or 30 seconds. Unfolding of the transducer may increase an elevation aperture of the transducer by at least 1.5 times relative to a conventional transducer of similar dimensions to the folded transducer. Thus if the dimensions of the transducer (with the SMP) are equal to a conventional array with a maximum elevation aperture of 2.2 mm, the unfolded transducer may have a maximum elevation aperture of 3 mm. Furthermore, a distance between transducer arrays of the transducer, as occupied by the SMP, may adversely affect image quality, e.g., as the distance increases, image quality is degraded. A distance occupied by the SMP in between transducer arrays may therefore be 5% or less than the elevation aperture. For example, if the elevation aperture is 3 mm, the SMP may extend no more than 0.15 mm between the transducer arrays.

In order to meet the criteria described above, various approaches to inducing conversion of an SMP implemented in a transducer between shapes may be contemplated. Examples of how the SMP may be activated to adjust between shapes are provided below.

### EXAMPLES

### Example 1

The SMP is initially in an unfolded, planar configuration, as shown in a second diagram 1100 in FIG. 11. The SMP is programmed by heating the SMP above a threshold temperature (Tₜᵣₐₙₛ in FIG. 11) that induces transformation of the SMP. By pressing the heated SMP against a support structure with a target shape, the SMP is programmed to have a first shape memory according to the support structure. As shown in FIG. 11, the first shape memory is U-shaped when formed against the support structure. Upon cooling below the threshold temperature the SMP adjusts to a second shape memory which is less bent than the first shape memory. The SMP is attached to a transducer and assembled in a deployable catheter. The SMP alternates between the first and second shape memories based exclusively on temperature.

### Example 2

An SMP is initially in an unfolded configuration. The SMP is coupled to a transducer and heated to a temperature above a threshold temperature that induces deformation of the SMP into a folded shape. The SMP is folded by applying a fixed strain to the SMP which is maintained as the temperature is cooled to below the threshold temperature. When the SMP is in the folded shape, a size of the transducer is reduced. The transducer, with the folded SMP, is assembled in a deployable catheter with the SMP in the first shape memory. The deployable catheter is inserted into a patient and, upon reaching a target site, heated to above the threshold temperature. The increased temperature causes the SMP to revert to the initial unfolded configuration, increasing the size of the transducer. The SMP is then subjected to a stimulus other than temperature which elicits a different physical response from the SMP. For example, a pH of a fluid in the deployable catheter is altered which causes the SMP to soften. The soft SMP is folded using an external mechanical force, such as catheter steering wires, reducing the size of the transducer array. The deployable catheter is then removed from the target site. The SMP is thus configured to respond to both changes in temperature and changes to another physical stimulus or to a chemical stimulus

### Example 3

An SMP is initially rigid and in an unfolded configuration. The SMP is coupled to a transducer and subjected to a first stimulus that causes the SMP to at transition to an at least partially pliable material. The first stimulus may any of a variety of stimuli, including, chemical, physical, etc. The SMP is adjusted to a folded shape by, for example, applying an external force and pressing the SMP against a structure to mold the shape of the SMP. The SMP is then exposed to a second stimulus, such as UV light, for example, to increase a rigidity of the SMP while in the folded shape. The second stimulus may be of a same type as the first stimulus, or different. The transducer, with the folded and rigid SMP, is assembled into a deployable catheter and inserted into a patient. Upon reaching a target site, the SMP is exposed to the first stimulus to soften the SMP. The SMP is unfolded using an external mechanical force, such as catheter steering wires, to increase an active area of the transducer and the SMP is fixed in the unfolded configuration by exposing the SMP to the second stimulus to harden the SMP. When scanning is complete, the SMP is subjected to the first stimulus to at least partially soften the SMP. The catheter steering wires are used to fold the SMP to decrease the size of the transducer. The deployable catheter is then removed from the target site. Thus the transducer size is varied based on a change in hardness of the SMP in response to one or more stimuli in combination with the external mechanical force.

### Example 4

A material spring property of an SMP is leveraged to adjust an active area of a transducer. The SMP is initially in an unfolded configuration and coupled to the transducer. The SMP is folded to adjust the transducer to a more compact formation with favorable dimensions for insertion into a patient and maintained in the folded shape by a constraint, e.g., a mechanical restraint such as a clip, a fastener, etc. The transducer is packaged into a deployable catheter with the SMP in the folded shape and the deployable catheter is inserted into a patient. Upon reaching a target site, the mechanical restraint may be released and the spring property of the SMP causes the SMP to unfold, increasing the active size of the transducer. When scanning is complete, the SMP is folded by applying an external mechanical force via, for example, catheter steering wires and secured in the folded shape by the mechanical restraint. The size of the transducer is reduced and the deployable catheter is removed from the target site. The SMP is therefore not programmed to respond to one or more stimuli. Instead, external mechanical forces are used in combination with the spring property of the SMP.

### Example 5

A SMP configured to respond to one or more stimuli by both folding, e.g., bending, and contracting, e.g., shrinking, along at least one dimension is coupled to a transducer. For example, the SMP is arranged in between transducer arrays of the transducer, thereby connecting the transducer arrays. The SMP is exposed to a first stimulus which induces a bending of the SMP into a folded configuration, reducing the size of the transducer. The transducer is packaged into a deployable catheter and inserted into a patient. Upon reaching a target site, the SMP is exposed to a second stimulus which may be of a same or different type as the first stimulus. The SMP unfolds in response to the second stimulus to increase the size of the transducer. Exposure to the second stimulus may also cause contraction of the SMP along, for example, a width of the transducer. As the SMP contracts, a distance between each of the transducer arrays, as occupied by the SMP, decreases. Alternatively, the SMP may be subjected to a third stimulus to trigger contraction of the SMP. Contraction of the SMP is described further below with reference to FIG. 12. When scanning is complete, the SMP is subjected to a fourth stimulus, which may be of a same or different type as the third stimulus, compelling expansion of the SMP along the same dimension as the contraction. The SMP is then exposed to the first stimulus to cause the SMP to fold and reduce the size of the transducer. The deployable catheter is then removed from the target site.

As described above, in order to maintain a performance of a transducer, a total distance between each transducer array of the transducer may be no more than a threshold percentage of an elevation aperture of the transducer, such as 5%. Thus minimizing the distance between the transducer arrays during data acquisition at the transducer is desirable. However, folding of the transducer array along an azimuth aperture, as shown in FIGS. 5, 6A, 7A, 7C, 8A, and 9A, may be a shape transition offering a lowest degree of complexity and easily initiated. To allow the transducer to be sufficiently folded along the azimuth aperture to reduce the transducer footprint, a spacing of the transducer arrays greater in total than the threshold percentage of the elevation aperture may be demanded.

The issue of maintaining the distance between transducer arrays below a threshold equivalent portion of the elevation aperture (e.g., 5%) may be addressed by utilizing a SMP configured to both fold and contract. The SMP may have a large deformation capability of, for example, up to 800%. By using an SMP adapted to contract along at least one dimension in response to a stimulus, the distance between transducer arrays may be decreased. For example, as shown in FIG. 12 in a third diagram 1200, a transducer 1250 has a first transducer array 1202 and a second transducer array 1204 spaced apart by a SMP 1206. The transducer 1250 is depicted in a first, folded configuration 1201, where an active area of the transducer 1250 is reduced.

Upon exposure to a first stimulus, S₁, the SMP 1206 transitions to a second, planar configuration 1203. The first stimulus may be any of the stimuli described above. An active area of the transducer 1250, e.g., a total surface area of the transducer 1250 facing a same direction along the y-axis, is doubled relative to the first configuration 1201. The first transducer array 1202 is spaced away from the second transducer array 1204 by the SMP 1206 which has a first width 1208 in the second configuration 1203, the width defined along the x-direction which may also be an elevation direction of the transducer 1250.

The SMP 1206 may be exposed to a second stimulus S₂, different from the first stimulus which may compel the SMP 1206 to shrink along the x-axis. A contraction of the SMP 1206 along the elevation direction transitions the transducer 1250 into a third configuration 1205. In the third configuration 1205, the SMP 1206 has a second width 1210 which is smaller than the first width 1208. For example, the second width 1210 may be 2% of the first width 1208 or between 1-10% of the first width 1208. The distance between the first and second transducer arrays 1202, 1204 is thus reduced. The transducer 1250 may be transitioned from the third configuration 1205 to the second configuration 1203 and from the second configuration 1205 to the first configuration 1201 by exposing the SMP 1206 to more than one stimulus.

It will be appreciated that the examples of shape transitions described above, e.g., bending and contracting, are non-limiting examples. Various other modes of shape change have been contemplated for use in a deployable catheter. For example, in addition bending and contracting, the SMP may curl, twist, and/or expand. The SMP may be configured to change shape via more than more mode depending on an applied stimulus and a desired level of complexity. Furthermore, variations in placement of a SMP relative to transducer arrays of a transducer are possible. For example, the SMP may be located outside of an active area of the transducer instead of in between each transducer array.

A method 1300 for implementing a SMP in a transducer for imaging via a deployable catheter is shown in FIG. 13. The SMP may be any type of SMP, including block copolymers, thermoplastics, linked cross-polymers, etc. The deployable catheter may be used for intracardiac echocardiography (ICE) imaging and may have a diameter of 2.67-3.3 mm, for example. Deployment of the catheter and activation of a shape transition of the SMP may be performed by at least one operator, such as a surgeon and or a technician. Alternatively, the deployment and activation may be conducted by an automated system, such as a robot.

At 1302 of the method, the transducer is fabricated. The transducer may include two or more transducer arrays, each transducer array coupled to an integrated circuit, and the transducer arrays connected to one another by sections of material formed from the SMP. Fabrication of the transducer may include attaching the SMP to the transducer arrays with an adhesive at 1304. In other examples, however, when the SMP has attenuating properties, such as when the SMP is configured as a matching layer, the SMP may be a part of the transducer arrays, e.g., integrated into the transducer arrays.

The fabrication process may further include adjusting the SMP to a first, folded shape at 1306. The SMP may be folded as shown in FIGS. 5, 6A, 7A, 8A, and 9A. The SMP may be folded to reduce a size of the transducer by programming the SMP to fold into the first shape in response to a first stimulus. For example, the first stimulus may be a temperature threshold that is equal to body temperature. When the SMP is at or below the temperature threshold, the SMP remains in the first shape. As another example, the first stimulus may be a humidity, e.g., a humidity of ambient air. Thus when the SMP is stored in air, the SMP remains in the first shape.

Alternatively, the SMP may be configured to soften in response to the first stimulus, rather than folding. In such an instance, the softened SMP may be folded into the first shape using an external mechanical force, such as a catheter steering wire. The SMP may then be held in the first shape by, for example, a mechanical constraint.

At 1308, the method includes assembling the deployable catheter in preparation for obtaining intravascular images. Assembling the deployable catheter may include, at 1310, packaging the transducer into a tip of the catheter. The transducer may be enclosed, along with integrated circuits and cables, within an outer housing of the catheter tip. In one example, the catheter tip may include a balloon and the transducer may be placed in the balloon. The balloon may be formed of an elastic, stretchable material that allows the balloon to be inflated. Assembling the deployable catheter may also include, at 1312, sterilizing and sealing the catheter in a protective outer coating or shell such as a plastic wrap or plastic housing to mitigate contamination of the sterilized catheter. The deployable catheter may then be shipped to a destination for use at a medical facility or stored on a shelf.

At 1314 of the method, the deployable catheter is used to obtain intravascular images of a patient. The protective outer coating is removed and the deployable catheter may be inserted intravenously into the patient through a small incision. The deployable catheter is fed through a vein or artery until the catheter tip reaches a target site. For example, to obtain ICE images, the catheter may travel through a coronary sinus of the patient and through a tricuspid valve into a right ventricle of the patient's heart. The catheter may be halted in the right ventricle to acquire images.

Prior to image acquisition, the SMP is adjusted to a second, unfolded shape at 1316. The SMP may be unfolded as shown in FIGS. FIGS. 5, 6B, 7B, 8B, and 9B. Unfolding the SMP may increase an active area of the transducer, enabling images with high resolution to be obtained in real-time. The SMP may be adjusted to the second shape by exposing the SMP to a second stimulus which may be of a same or different type as the first stimulus. For example, the catheter may include a heating unit which may be activated to heat the SMP above the threshold temperature. The elevated temperature may induce unfolding of the SMP and the temperature may be maintained above the threshold temperature during image acquisition. As another example, the second stimulus may be a change in humidity. The balloon in which the transducer may be enclosed may be filled with a fluid, such as water. The increase in humidity may trigger unfolding of the SMP.

Alternatively, when the SMP is softened in response to the first stimulus and maintained in the first shape by the mechanical constraint, the mechanical constraint may be removed at the target site and the SMP returns to the unfolded shape as a result of a spring property of the material. The SMP, in the second shape, may harden in response to exposure to the second stimulus.

At 1318, images are acquired by the transducer. Imaging data may be collected, processed, and displayed as described above, with reference to FIG. 1. Activation of the transducer may be initiated by the operator pressing a button that delivers a current to the transducer from an electrical source. Upon completion of image acquisition, the SMP is adjusted to the first shape at 1320. For example, the SMP may be exposed to the first stimulus which, in one example, may include deactivating the heating unit of the deployable catheter and cooling the temperature to the threshold temperature. As another example, the fluid in the balloon may be drained and the balloon purged with a gas to decrease the humidity. The folding of the SMP returns the transducer to a decreased size.

Alternatively, exposing the SMP to the first stimulus may soften the SMP and the transducer may be folded using the external mechanical force. The mechanical constraint may be applied to the folded transducer to maintain the transducer in the first shape.

At 1322, the method includes removing the deployable catheter tip from the target site. The deployable catheter may be removed from the patient entirely or directed to another target site for further data collection. The method then ends.

In this way, a transducer for a deployable catheter may readily pass intravenously through a patient and provide images with enhanced field of view, resolution, penetration, image update rate. Transducer arrays of the transducer may be linked to one another by a SMP and the transducer may transition between at least a first, folded shape and a second, unfolded shape as a result of a response of SMP to stimuli. The transducer may be adjusted to the first shape by exposure to a first stimulus. Folding of the transducer decreases a size of the transducer, allowing the transducer to pass through arteries and veins without hindrance. The transducer may transition to the second shape in response to exposure of the SMP to a second stimulus. Unfolding the transducer increases a size, and therefore and active area of the transducer, allowing a performance of the transducer to be increased. The larger overall size of the transducer also enables, less complex, lower cost fabrication of the deployable catheter. Furthermore, by configuring the SMP with two-way shape memory, the transducer may be folded at the target site and returned to smaller dimensions to allow the deployable catheter to be deployed at another site and/or easily extracted.

The technical effect of implementing the SMP in the transducer for the deployable catheter is that the resolution, penetration, and data acquisition speed of the transducer is increased while

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The disclosure also provides support for a deployable invasive device comprising: a transducer, including a plurality of transducer arrays spaced apart by a shape memory material, configured to transition between a first folded shape and a second unfolded shape. In a first example of the system, the shape memory material is a shape memory polymer. In a second example of the system, optionally including the first example, the shape memory material is a shape memory polymer with two-way shape memory. In a third example of the system, optionally including the first and second examples, each transducer array of the plurality of transducer arrays is linked to adjacent transducer arrays by the shape memory material and wherein the shape memory material is configured to couple to the transducer by at least one of attaching to edges of the plurality of transducer arrays and forming a layer extending entirely across the transducer, below the plurality of transducer arrays. In a fourth example of the system, optionally including the first through third examples, an integrated circuit is coupled to each transducer array of the plurality of transducer arrays, and wherein the plurality of transducer arrays are spaced apart in both the folded and unfolded configuration. In a fifth example of the system, optionally including the first through fourth examples, a length or a width of an active area of the second unfolded shape is larger than a length or a width of an active area of the first folded shape, respectively. In a sixth example of the system, optionally including the first through fifth examples, when in the first folded shape, the shape memory material is bent in between each of the plurality of transducer arrays and wherein the plurality of transducer arrays are stacked along a vertical axis of the transducer and spaced apart in both the first folded shape and second unfolded shape. In a seventh example of the system, optionally including the first through sixth examples, when the transducer is in the second unfolded shape, the shape memory material is less bent than when the transducer is in the first folded shape. In an eighth example of the system, optionally including the first through seventh examples, the shape memory material is configured to undergo more than one type of shape transition and wherein the more than one type of shape transition includes bending and shrinking.

The disclosure also provides support for a transducer for an imaging catheter comprising: a first transducer array, and a second transducer array coupled to the first transducer array by a shape memory polymer (SMP), wherein the SMP is configured to change shape to transition the transducer to a first geometry in response to a first stimulus and to unfold to a second geometry in response to a second stimulus. In a first example of the system, the SMP is arranged between the first transducer array and the second transducer array and configured to bend or fold when the transducer is in the first geometry. In a second example of the system, optionally including the first example, the first transducer array is pivoted through a first rotational direction relative to the second transducer array and stacked over the second transducer array when the transducer is in the first geometry. In a third example of the system, optionally including the first and second examples, the first transducer array is pivoted in a second rotational direction, opposite of the first rotational direction, relative to the second transducer array when the transducer is adjusted from the first geometry to the second geometry. In a fourth example of the system, optionally including the first through third examples, the first transducer array and the second transducer array are co-planar in the second geometry and an active area of the transducer is increased when the transducer is adjusted from the first geometry to the second geometry. In a fifth example of the system, optionally including the first through fourth examples, the system further comprises: a third transducer array coupled to the second transducer array by the SMP at an opposite side of the second transducer array from the first transducer array and wherein the SMP is arranged between the second transducer array and the third transducer array. In a sixth example of the system, optionally including the first through fifth examples, the first transducer array is pivoted between through a first rotational direction relative to the second transducer array and stacked over the second transducer array and the third transducer array is pivoted through a second rotational direction, opposite of the first rotational direction, relative to the second transducer array and stacked under the second transducer array when the transducer is in the first geometry. In a seventh example of the system, optionally including the first through sixth examples, the first transducer array is pivoted between through the second rotational direction relative to the second transducer array and the third transducer array is pivoted through the first rotational direction relative to the second transducer array when the transducer is adjusted from the first geometry to the second geometry. In an eighth example of the system, optionally including the first through seventh examples, the first, the second, and the third transducer arrays are co-planar when the transducer is in the second geometry and an active area of the transducer is increased when the transducer is adjusted from the first geometry to the second geometry.

The disclosure also provides support for a method for a deployable catheter comprising: fabricating a transducer with a shape memory polymer (SMP), and responsive to application of a first stimulus, folding the SMP to reduce a footprint of the transducer, and responsive to application of a second stimulus, unfolding the SMP to increase the footprint of the transducer. In a first example of the method, applying the first stimulus and the second stimulus includes exposing the SMP to any combination of physical, chemical, and biological stimuli.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A deployable invasive device comprising:
a transducer, including a plurality of transducer arrays spaced apart by a shape memory material, configured to transition between a first folded shape and a second unfolded shape.

2. The deployable invasive device of claim 1, wherein the shape memory material is a shape memory polymer.

3. The deployable invasive device of claim 1, wherein the shape memory material is a shape memory polymer with two-way shape memory.

4. The deployable invasive device of claim 1, wherein each transducer array of the plurality of transducer arrays is linked to adjacent transducer arrays by the shape memory material and wherein the shape memory material is configured to couple to the transducer by at least one of attaching to edges of the plurality of transducer arrays and forming a layer extending entirely across the transducer, below the plurality of transducer arrays.

5. The deployable invasive device of claim 1, wherein an integrated circuit is coupled to each transducer array of the plurality of transducer arrays, and wherein the arrays are spaced apart in both the first folded shape and the second unfolded shape.

6. The deployable invasive device of claim 1, wherein a length or a width of an active area of the second unfolded shape is larger than a length or a width of an active area of the first folded shape, respectively.

7. The deployable invasive device of claim 1, wherein when in the first folded shape, the shape memory material is bent in between each of the plurality of transducer arrays and wherein the plurality of transducer arrays are stacked along a vertical axis of the transducer and spaced apart in both the first folded shape and the second unfolded shape.

8. The deployable invasive device of claim 1, wherein when the transducer is in the second unfolded shape, the shape memory material is less bent than when the transducer is in the first folded shape.

9. The deployable invasive device of claim 1, wherein the shape memory material is configured to undergo more than one type of shape transition and wherein the more than one type of shape transition includes bending and shrinking.

10. A transducer for an imaging catheter comprising:
a first transducer array; and
a second transducer array coupled to the first transducer array by a shape memory polymer (SMP), wherein the SMP is configured to change shape to transition the transducer to a first geometry in response to a first stimulus and to unfold to a second geometry in response to a second stimulus.

11. The transducer of claim 10, wherein the SMP is arranged between the first transducer array and the second transducer array and configured to bend or fold when the transducer is in the first geometry.

12. The transducer of claim 10, wherein the first transducer array is pivoted through a first rotational direction relative to the second transducer array and stacked over the second transducer array when the transducer is in the first geometry.

13. The transducer of claim 12, wherein the first transducer array is pivoted in a second rotational direction, opposite of the first rotational direction, relative to the second transducer array when the transducer is adjusted from the first geometry to the second geometry.

14. The transducer of claim 13, wherein the first transducer array and the second transducer array are co-planar in the second geometry and an active area of the transducer is increased when the transducer is adjusted from the first geometry to the second geometry.

15. The transducer of claim 10, further comprising a third transducer array coupled to the second transducer array by the SMP at an opposite side of the second transducer array from the first transducer array and wherein the SMP is arranged between the second transducer array and the third transducer array.

16. The transducer of claim 15, wherein the first transducer array is pivoted between through a first rotational direction relative to the second transducer array and stacked over the second transducer array and the third transducer array is pivoted through a second rotational direction, opposite of the first rotational direction, relative to the second transducer array and stacked under the second transducer array when the transducer is in the first geometry.

17. The transducer of claim 16, wherein the first transducer array is pivoted between through the second rotational direction relative to the second transducer array and the third transducer array is pivoted through the first rotational direction relative to the second transducer array when the transducer is adjusted from the first geometry to the second geometry.

18. The transducer of claim 17 wherein the first, the second, and the third transducer arrays are co-planar when the transducer is in the second geometry and an active area of the transducer is increased when the transducer is adjusted from the first geometry to the second geometry.

19. A method for a deployable catheter comprising:
fabricating a transducer with a shape memory polymer (SMP); and
responsive to application of a first stimulus;
folding the SMP to reduce a footprint of the transducer; and
responsive to application of a second stimulus;
unfolding the SMP to increase the footprint of the transducer.

20. The method of claim 19, wherein applying the first and the second stimuli includes exposing the SMP to any combination of physical, chemical, and biological stimuli.
